(19) United States
(12) Patent Application Publication
(10) Pub. No.: US 2012/0022113 A1
(43) Pub. Date: Jan. 26, 2012

(54) METHODS FOR SUPPRESSING TIPDC TRAFFICKING OR ACCUMULATION AND PREVENTING HYPERCYTOKINEMIA

(76) Inventors: Jerry R. Aldridge, Jr., Cordova, TN (US); Carson E. Moseley, Greenville, AL (US)

(21) Appl. No.: 13/145,119

(22) PCT Filed: Jan. 11, 2010
(86) PCT No.: PCT/US10/20608

§ 371 Date: Jul. 19, 2011

Related U.S. Application Data
(60) Provisional application No. 61/146,103, filed on Jan. 21, 2009.

(51) Int. Cl. A61K 31/4439; A61P 37/02; A61P 31/16

(52) U.S. Cl. 514/342

(57) ABSTRACT

The present invention features methods for preventing or ameliorating hypercytokinemia or preventing lethality-associated with infection by highly pathogenic strains of influenza virus by suppressing TNF-α/Inducible nitric oxide synthase-Producing dendritic cell trafficking or accumulation. In particular embodiments, the expression MyD88, CCR2, MCP-I or MCP-3 is targeted.

This application claims benefit of U.S. Provisional Patent Application Ser. No. 61/146,103, filed Jan. 21, 2009, the content of which is incorporated herein by reference in its entirety.

### INTRODUCTION

### Background of the Invention

Epidemics and pandemics of influenza vary in pathogenicity. On average, the annual influenza epidemic, also referred to as seasonal influenza, results in over 35,000 deaths in the United States alone Meltzer, et al. (1999) Emerging Infectious Diseases 5(5):659. In contrast, the most well documented highly pathogenic pandemic, the 1918 Spanish flu, resulted in the deaths of 40-50 million people worldwide (Horimoto & Kawaoka (2005) Nature Rev. 3(8):591; Loo & Gale, Jr. (2007) Nature 445(7125):267; Palese (2004) Nature Med. 10(12 Suppl):S82; Smith (2007) Nature 445(7125):237; Taubenberger, et al. (2005) Nature 437(7060)889; Tumpey, et al. (2005) Science 310(5745):77). In 1996, H5N1 influenza emerged from its zoonotic source, aquatic birds, and as of September 2008 the WHO reported 387 laboratory-confirmed human infections with 245 resulting in death (63% mortality rate). Interestingly, while seasonal influenza viruses generally cause mortality in either young or old individuals, highly lethal strains including the currently circulating H5N1 viruses induce a disproportionably high death rate in the most immunocompetent individuals (Tumpey, et al. (2005) supra).

### SUMMARY OF THE INVENTION

The present invention features a method for preventing or ameliorating hypercytokinemia and a method for reducing severity or protecting against lethality of an influenza A virus infection by administering to a subject in need of treatment an agent that suppresses TNF-α/Inducible nitric oxide synthase-Producing dendritic cell (tipDC) trafficking or accumulation. In certain embodiments of these methods, the agent reduces the expression or activity of MyD88, CCR0, MCP-1 or MCP-3. In particular embodiments, the agent is a peroxisome proliferator-activated receptor-γ (PPARγ) agonist. In specific embodiments, the agent is a thiazolidinedione such as rosiglitazone, pioglitazone, troglitazone, rivoglitazone, ciglitazone, RS5444 or MCC-555.

A method for suppressing tipDC trafficking or accumulation is also provided. In accordance with this method, a subject in need of treatment is administered a PPARγ agonist thereby suppressing tipDC trafficking or accumulation. In particular embodiments, the agonist is a thiazolidinedione. In specific embodiments, the thiazolidinedione is rosiglitazone, pioglitazone, troglitazone, rivoglitazone, ciglitazone, RS5444 or MCC-555.

### BRIEF DESCRIPTION OF THE DRAWINGS

FIG. 1 shows increased survival after influenza infection using peroxisome proliferator-activated receptor-γ (PPARγ) agonist pioglitazone or rosiglitazone, and/or the AMP-kinase agonist AICAR.

### DETAILED DESCRIPTION OF THE INVENTION

A dysregulated immune response, commonly referred to as a cytokine storm or hypercytokinemia, has been shown to be associated with elevated levels of proinflammatory cytokines, both in vitro and in vivo (Kash, et al. (2006 Nature 443 (7111):578; Kobasa, et al. (2007) Nature 445(7125):319; Kobasa, et al. (2004) Nature 431(7009):703; Loo & Gale, Jr. (2007) supra; Smith (2007) supra; Chan, et al. (2005) Respiratory Research 6:135; Cheung, et al. (2002) Lancet 360(9348):1831). It has now been shown that a subset of dendritic cells (DC), previously described as TNF-α/Inducible nitric oxide synthase-Producing DC (tipDC) (Serbina, et al. (2003) Immunity 19(1):59), accumulate to significantly higher numbers in lethal influenza infections as compared to sublethal infections. It was observed that elimination of these cells was equally detrimental to the host because tipDCs are critical for complete expansion of the cytotoxic T-cell response. In addition, it was found that partial suppression of tipDC trafficking through treatment with a peroxisome proliferator-activated receptor-γ (PPARγ) agonist provides protection from lethal influenza challenge.

Accordingly, the present invention embraces methods for suppressing tipDC trafficking to or accumulation in affected tissue and preventing or ameliorating hypercytokinemia. In accordance with this invention, an effective amount of an agent that suppresses tipDC trafficking or accumulation is administered to a subject in need of such treatment, tipDC trafficking or accumulation is reduced or suppressed and hypercytokinemia is prevented or ameliorated.

As is conventional in the art, hypercytokinemia is a condition in which the concentration of cytokines in the blood is elevated. These cytokines include, e.g., IL-1β (interleukin-1 beta), IL-6 (interleukin-6), IL-10 (interleukin-10), INF-γ (interferon-gamma), TNF-α (tumor necrosis factor-alpha), GM-CSF (granulocyte macrophage-colony stimulating factor), IL-8 (interleukin-8) and MCP-1 (macrophage chemoattractant protein-1). In addition, as the results presented herein indicate, hypercytokinemia is also associated with an increase in tipDC trafficking to or accumulation in affected tissue. Accordingly, the prevention or amelioration of hypercytokinemia would result in a measurable decrease in the levels of one or more cytokines (e.g., MCP-1 or MCP-3) and/or a reduction in tipDC trafficking to or accumulation in affected tissue.

Hypercytokinemia can occur in a number of infectious and non-infectious diseases including graft versus host disease (GVHD), wherein blood, skin, liver, or the intestinal tissue of the recipient are adversely affected; adult respiratory distress syndrome (ARDS); sepsis; lethal influenza infection; smallpox; systemic inflammatory response syndrome (SIRS); multiple organ failure; and toxic shock syndrome, as well as in cancer chemotherapy patients. Thus, a subject having or at risk of having one or more of these diseases or conditions could benefit from the suppression of tipDC trafficking to or accumulation in the relevant affected tissues.

Subjects treatable in accordance with the present invention include humans as well as other animals. Such animals can be industrial animals, companion animals or experimental animals. Industrial animals are animals which need to be fed for industrial purposes, such as bovines, equines, swines, caprines, ovines and other livestock, chickens, ducks, quail, turkeys, ostriches and other fowl. Companion animals are dogs, cats, marmosets, cage birds, hamsters, and other pet animals, while experimental animals are rats, guinea pigs, beagle dogs, miniature pigs, rhesus monkeys, macaques and other animals used for research in medical, biological, agricultural, pharmaceutical and other fields.

TipDCs are known in the art as a monocyte-derived, inflammatory dendritic cell population characterized as having an intermediate level of CD11c and CD11b, a high level of Mac3 (CD11b^{int}/CD11c^{int}/Mac-3^{high}) and producing large amounts of tumor necrosis factor (TNF) and inducible nitric oxide synthase (iNOS) (Serbina, et al. (2003) supra). These cells have been characterized using mice infected with the intracellular bacteria Listeria monocytogenes (Serbina & Pamer (2006) Nat. Immunol. 7:311-317; Serbina, et al. (2003) supra; Sunderkotter, et al. (2004) J. Immunol. 172:4410-4417; Drevets, et al. (2004) J. Immunol. 172:4418-4424). Using this model system, it was shown that Gr1⁺ (Ly6C⁺) blood monocytes egress massively from bone marrow to the bloodstream in a CCR2-dependent fashion, differentiate via a MyD88-dependent mechanism into tipDCs that produce TNF-α and nitric oxide, and upregulate major histocompatibility complex-II antigens, CD80, CD86 and CD11c (Serbina, et al. (2006) supra; Serbina, et al. (2003) supra; Tsou, et al. (2007) J. Clin. Invest. 117:902-909). Accordingly, the present invention embraces targeting CCR2 or its ligands, or MyD88 to reduce or suppress the maturation, activation, trafficking or accumulation of tipDCs.

In one embodiment, MyD88 (myeloid differentiation factor 88) expression or activity is inhibited or decreased. In another embodiment, CCR2 (chemokine (C-C motif) receptor or CD192) expression or activity is inhibited or decreased. In so far as MCP-1 and MCP-3 are ligands of CCR2 (Jia, et al. (2008) J. Immunol. 180:6846-6853), further embodiments embrace inhibiting the expression or activity of MCP-1 (Monocyte chemoattractant protein 1, also known as Chemokine (C-C motif) ligand 2, CCL2) and/or MCP-3 (Monocyte chemoattractant protein 3). When inhibiting the expression or activity of MyD88, CCR2, MCP-1, or MCP-3, it is desirable that MyD88, CCR2, MCP-1, or MCP-3 expression or activity is inhibited by at least 10%, 20%, 30%, 40%, 50%, 60%, 70%, 80%, 90%, or 95% when compared to untreated cells or tissues. In one embodiment, MyD88, CCR2, MCP-1, or MCP-3 expression is inhibited by an agent that binds to a nucleic acid molecule encoding MyD88, CCR2, MCP-1, or MCP-3 thereby increasing the degradation or decreasing expression of said nucleic acid molecule. In another embodiment, MyD88, CCR2, MCP-1, or MCP-3 activity is inhibited by an agent that binds to MyD88, CCR2, MCP-1, or MCP-3 thereby inhibiting the activity thereof. As a result of said inhibition, tipDC trafficking or accumulation is suppressed in the affected tissue by at least 10%, 20%, 30%, 40%, 50%, 60%, 70%, 80%, 90%, or 95% when compared to untreated cells or tissues. However, in specific embodiments, tipDC trafficking or accumulation is not completely (i.e., 100%) suppressed.

The reduction in MyD88, CCR2, MCP-1, or MCP-3 expression or activity can be monitored using any conventional methodology which measures mRNA levels (e.g., northern blot analysis and RT-PCR), protein levels (e.g., western blot analysis), or activity (e.g., expression of downstream proteins). Likewise, a reduction in tipDC trafficking to or accumulation in affected tissue can be measured by conventional methods, e.g., flow cytometry analysis. The affected tissue can include the site of an infection, tumor or inflammation. By way of illustration, the affected tissue in an influenza A infection is the respiratory tract.

Agents useful for inhibiting the expression or activity of MyD88 have been described. These include, e.g., ST2 effector molecule, which sequesters MyD88 (Brint, et al. (2004) Nat. Immunol. 5:373-379), vaccinia virus ORFs A46R and A52R, which mimic the dominant-negative effect of a truncated version of MyD88 (Bowie, et al. (2000) Proc. Acad. Natl. Sci. USA 97:10162-10167), hydrocinnamoyl-L-valyl pyrrolidine, which is a low molecular weight MyD88 mimic (Bartfai, et al. (2003) Proc. Natl. Acad. Sci. USA 100:7971-7976), or an anti-MyD88 antagonistic antibody. In addition, siRNA (Zhu, et al. (2008) Transplant Proc. 40(5):1625-8; Yu, et al. (2008) Mol Cell Biochem. 317(1-2):143-50) and antisense oligonucleotides (ISIS 337846; Vickers, et al. (2006) J. Immunol. 176(6):3652-61) have been shown to inhibit the expression of MyD88.

Agents useful for inhibiting the expression or activity of CCR2 have also been described in the art. These include, e.g., simvastatin, (Yin, et al. (2007) J. Heart Lung Transplant. 26:485-493), oxidized LDL (Han, et al. (2000) J. Clin. Invest. 106(6):793-802), and PPARγ agonists (Ishibashi, et al. (2002) Hypertension 40(5):687-93; Tanaka, et al. (2005) Eur. J. Pharmacol. 508(1-3):255-65), which down-regulate expression of CCR2. In addition, substituted dipiperidine alcohols (Xia, et al. (2008) Bioorg. Med. Chem. Lett. 18:3562-3564), anti-CCR2 antibodies (WO 2007/147026), INCB3344 (Brodmerkel, et al. (2005) J. Immunol. 175:5370-5378), and 3-amino-1-alkyl-cyclopentane carboxamides (Pasternak, et al. (2008) Bioorg. Med. Chem. Lett. 18:1374-1377) have been shown to antagonize CCR2.

Bicyclic pyrrole derivatives have been described for use in inhibiting the activity of MCP-1 (U.S. Pat. No. 6,479,527). In addition, MCP-1(9-76) (Gong (1997) J. Exp. Med. 186: 131-137); 7ND (Zhang, et al. (1995) Mol. Cell. Biol. 15:4851-4855) and an anti-MCP-1 antibody (Li, et al. (2005) Am. J. Pathol. 167:637-49) have been shown to antagonize MCP-1. Moreover, MCP-3 (10-76) is a known antagonist of MCP-3 (Gong, et al. (1996) J. Biol. Chem. 271:10521-10527).

In particular embodiments, tipDC trafficking or accumulation is suppressed by targeting CCR2 with a PPARγ agonist. PPARγ agonists are known in the art and include, e.g., 15-deoxy-Δ^{12,14}-prostaglandin J₂ and azelaoyl PAF, which are naturally derived agonists (Lehmann, et al. (1995) J. Biol. Chem. 270:12953-12956; Cantello, et al. (1994) J. Med. Chem. 37:3977-3985). A class of well-known synthetic PPARγ agonists is the thiazolidinediones or TZDs. Members of the thiazolidinedione class of compounds have the chemical structure depicted in Formula I.

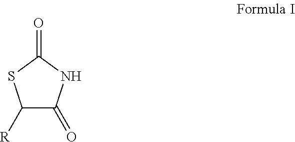

Chemically, the members of this class are derivatives of the parent compound thiazolidinedione, and in specific embodiments include rosiglitazone (BRL 49653), pioglitazone, troglitazone, rivoglitazone, ciglitazone, RS5444 and MCC-555, which is a structural homolog of rosiglitazone. See, Chou, et al. (2007) Mol. Cancer. Res. 5(6):523-30; Chen, et al. (2006) Mol. Cell. Endocrinol. 251:17-32.

Additional agents of use in the methods of this invention can be identified by screening assays. An example of such a screening assay would involve exposing a host (e.g., a mammal such as a mouse or rat) to a test agent, inducing hypercytokinemia in the subject (e.g., exposing the animal to a lethal viral infection) and monitoring the ability of the test agent to reduce or suppress trafficking or accumulation of tipDCs, prevent hypercytokinemia, or provide protection against lethality induced by viral infection. When the outcome of the screening assay includes monitoring trafficking or accumulation of tipDCs, it is particularly desirable that the test agent does not completely eliminate accumulation of the tipDCs.

Agents which can be screened for the desired activity can be obtained from a wide variety of sources including libraries of synthetic or natural compounds. A library can comprise either collections of pure agents or collections of agent mixtures. Examples of pure agents include, but are not limited to, antibodies, peptides, peptide aptamers, polypeptides, oligonucleotides, carbohydrates, fatty acids, steroids, purines, pyrimidines, lipids, synthetic or semi-synthetic chemicals, and purified natural products, as well as derivatives or structural analogs of the agents specifically disclosed herein. Examples of agent mixtures include, but are not limited to, extracts of prokaryotic or eukaryotic cells and tissues, as well as fermentation broths and cell or tissue culture supernatants. In the case of agent mixtures, one may not only identify those crude mixtures that possess the desired activity, but also monitor purification of the active component from the mixture for characterization and development as a therapeutic drug. In particular, the mixture so identified can be sequentially fractionated by methods commonly known to those skilled in the art which may include, but are not limited to, precipitation, centrifugation, filtration, ultrafiltration, selective digestion, extraction, chromatography, electrophoresis or complex formation. Each resulting subfraction can be assayed for the desired activity using the original assay until a pure, biologically active agent is obtained.

In particular embodiments of this invention, agents disclosed herein, which inhibit the expression or activity of MyD88, CCR2, MCP-1, or MCP-3 thereby suppressing tipDC trafficking or accumulation reduce the severity of, or protect against lethality associated with, an infection by highly pathogenic influenza virus strains in a subject having or at risk of a lethal influenza infection (e.g., a subject exposed to a lethal influenza A virus such as H5N1). Subjects benefiting from treatment with an agent disclosed herein will exhibit a reduction in the severity of the symptoms of an influenza virus infection and/or will recover more quickly than a subject not treated with the agent. In particular embodiments, an agent of the invention will reduce mortality by at least 50% in a population of subjects infected with a lethal influenza virus infection (e.g., an H5N1 infection).

When used therapeutically, agents disclosed herein (including those identified in screening assays) are typically provided in the form of a pharmaceutical composition that contains the agent and a pharmaceutically acceptable carrier. A pharmaceutically acceptable carrier is a material useful for the purpose of administering the medicament, which is preferably sterile and non-toxic, and can be solid, liquid, or gaseous materials, which is otherwise inert and medically acceptable, and is compatible with the active ingredients. A generally recognized compendium of methods and ingredients of pharmaceutical compositions is Remington: The Science and Practice of Pharmacy, Alfonso R. Gennaro, editor, 20th ed. Lippincott Williams & Wilkins: Philadelphia, Pa., 2000.

A pharmaceutical composition can contain other active ingredients such as preservatives. A pharmaceutical composition can take the form of a solution, emulsion, suspension, ointment, cream, granule, powder, drops, spray, tablet, capsule, sachet, lozenge, ampoule, pessary, or suppository. It can be administered by continuous or intermittent infusion, parenterally, intramuscularly, subcutaneously, intravenously, intra-arterially, intrathecally, intraarticularly, transdermally, orally, bucally, intranasally, as a suppository or pessary, topically, as an aerosol, spray, or drops, depending upon whether the preparation is used to treat an internal or external condition or disease. Such administration can be accompanied by pharmacologic studies to determine the optimal dose and schedule and would be within the skill of the ordinary practitioner.

The invention is described in greater detail by the following non-limiting examples.

### Example 1

### Materials and Methods

Viruses. The H1N1 A/Puerto Rico/8/34 (PR8) and H3N2 A/Aichi/68 (x-31) influenza A viruses were obtained from the St. Jude Children's Research Hospital repository. The x-31 double knockout (DKO) virus with mutated, non-responder D^{b}NP₃₆₆₋₃₇₄ and D^{b}PA₂₂₄₋₂₃₃ peptides was generated earlier using the eight-plasmid reverse-genetics system (Webby, et al. (2003) Proc. Natl. Acad. Sci. USA 100(12):7235; Hoffmann, et al. (2000) Proc. Natl. Acad. Sci. USA 97(11):6108). The H5N1 A/Hong Kong/213/03 (HK213) and H5N1 A/Vietnam/1203/2004 (VN1203) viruses were obtained through the WHO Global Influenza Surveillance Network. Stocks were propagated by allantoic inoculation of 10-day old embryonated chicken's eggs and virus titers were determined as plaque forming units (pfu) on MDCK monolayers. The experiments with H5N1 viruses were conducted in a USDA-approved Biosafety level (BSL) 3+ containment facility.

Mice. Wild-type C57BL/6J (B6) and CCR2^{-/-} mice were purchased from The Jackson Laboratory (Bar Harbor Me.), and were housed under SPF conditions. All mice in this study were used according to protocols approved by the Institutional Animal Care and Use Committee at St. Jude Children's Research Hospital.

Viral Infection and Sample Collection. Naïve mice were anesthetized by intra-peritoneal (i.p.) injection with avertin (2,2,2-tribromoethanol) prior to i.n. challenge with the appropriate dose of virus diluted in 30 μl of sterile, endotoxin-free phosphate-buffered saline (PBS). Animals were weighed prior to infection, then monitored daily for weight loss as a measure of morbidity, or euthanized at the appropriate time point for sample collection. Bronchoalveolar lavage (BAL) fluid was collected in three 1 ml washes with Hank's Buffered Salt Solution (HESS). Cells were pelleted by light centrifugation and total numbers per BAL sample were determined using a Coulter Counter (IG Instrumenten Gesellschaft AG).

Flow-Cytometric Analysis. The BAL cells were stained with the appropriate combination of FITC-labeled anti-Ly6g, phycoerythrin (PE)-labeled anti-Class II, PE-Cy5-labeled anti-CD11c, ALEXA Fluor 647-labeled anti-Lytic, allophycocyanin (APC)-Cy7-labeled anti-CD11b, FITC-labeled anti-CD4, PE-labeled anti-NK1.1, APC-labeled αβTCR, or APC-Cy7-labeled anti-CD8 (eBiosciences, San Diego, Calif.) after blocking of the Fc receptor with anti-CD32/CD16 (BD Biosciences, San Diego, Calif.) at 4° C.

For CD8⁺ T-cell analysis, cells were stained with tetramers specific for the D^{b}PA₂₂₄₋₂₃₃, K^{b}PB1₇₀₃₋₇₁₁, or D^{b}PB1-F2₆₂₋₇₀ epitopes for 1 hour at room temperature prior to staining with the monoclonal antibodies (mAbs). Data were acquired using either a FACSCALIBUR or FACSSCAN (BD Biosciences) and analyzed using FloJo (Tree Star Inc., Ashland, Oreg.).

Cytokine and Chemokine Quantification. Mouse cytokines and chemokines in the cell-free BAL fluid were measured using the BIORAD multiplex assay.

Virus Titer Determination. Cell-free BAL fluid samples were titered by plaque assay on MDCK cells. Near confluent 25 cm² MDCK cell monolayers were infected with 1 ml of six ten-fold dilutions of BAL fluid for 1 hour at 37° C., then washed with PBS prior to adding 3 ml MEM containing 1 mg/ml L-1-tosylamido-2-phenylethyl chloromethyl ketone-treated trypsin (Worthington Biochemical Corporation, Lakewood, N.J.) and 0.9% agarose. Cultures were incubated at 37° C., 5% CO₂ for 72 hours. Plaques were visualized with crystal violet.

T-Cell Hybridoma Stimulation by tipDCs. Wild-type B6 mice were infected with 10⁵ pfu of x-31. On day 6 after infection, BAL fluid was collected and tipDCs were purified by cell sorting (MOFLO; Beckman Coulter, Fullerton, Calif.). The tipDCs (˜1×10⁴) were co-cultured in 96-well plates with 2×10⁴ D^{b}PB1-F2₆₂-specific hybridoma cells (line F2-54) for 24 hours. The F2-54 hybridoma line reacts positively by IL-2 production with the D^{b}PB1-F2₆₂ epitope but negatively with D^{b}PA₂₂₄, K^{b}PB1₇₀₃, K^{b}NS2₁₁₄ and D^{b}NP₃₆₆. At the same time, a standard curve was generated by pulsing uninfected splenocytes with serially diluted PB1-F2₆₂ peptide, then co-culture with the F2-54 hybridoma. IL-2 production, which in this assay correlates directly to the MHC class I expression on the APC cell surface, was measured by ELISA using purified anti-IL-2 and biotin-anti-IL-2 (BD Pharmingen, Inc., San Diego, Calif.) antibodies following the manufacturer's directions. The concentration of epitope was calculated based on IL-2 production from the standard curve.

tipDC Transfer. The B6 mice were infected i.n. with 10⁵ pfu of either the intact x-31 or DKO virus disrupted for the D^{b}NP₃₆₆₋₃₇₄ and D^{b}PA₂₂₄₋₂₃₃ epitopes. On day 6 after infection, tipDCs were isolated from the BAL fluid using a MOFLO to greater than 95% purity. The CCR2^{-/-} animals were infected i.n. with 10⁵ pfu of x-31. On day 1 after infection, 10⁴ tipDCs, purified from animals given either x-31 or DKO, were transferred intratracheally (i.t.) into CCR2^{-/-} animals. Six days after tipDC transfer (day 7 after infection) BAL fluid was collected and cells were stained with tetramers specific for the D^{b}NP₃₆₆₋₃₇₄ and D^{b}PA₂₂₄₋₂₃₃ epitopes at room temperature for 1 hour. After blocking the Fc receptor with anti-CD32/CD16 (BD Biosciences) at 4° C., cells were then stained with APC-Cy7 labeled anti-CD8 (eBiosciences) and analyzed by flow cytometry (FACSSCAN; BD Pharmingen). Total cell numbers per BAL sample were determined using a Coulter Counter (IG Instrumenten Gesellschaft AG).

Pioglitazone Treatment. B6 mice were treated with 60 mg/kg of pioglitazone (suspended in 100 μl of PBS) via oral gavage beginning on day 3 before infection and continued daily thereafter. The control mice were given 100 μl of PBS and both groups were challenged with 5 MLD₅₀ of PR8. Weight loss was monitored daily as a measure of morbidity. On days 3 and 6 after infection, groups of mice from the treatment and control groups were euthanized. The BAL fluid was collected in three 1 ml washes with Hank's Buffered Salt Solution (HBSS) and cells were isolated via light centrifugation. The total cell count per BAL was determined using a Coulter Counter and the cells were analyzed by flow cytometry. Virus titers in BAL fluid were determined by plaque assay. Cytokines/chemokines were quantified using the BIORAD multiplex assay, except for MCP-3, which was measured by ELISA (BenderMed Systems, Burlingame, Calif.).

Statistical Analysis. Data are presented as mean±s.e.m. Time course data, i.e., cell counts and cytokine/chemokine expression, were analyzed with multiple regression. When there was a significant interaction effect, the simple effect of virus or treatment was investigated by performing ANOVA for each time point. This analysis accounted for multiple-comparisons by using the mean square error from the regression analysis to calculate the F-statistic. If the interaction effect was not significant, then model simplification was performed to find the minimal adequate model. Similar techniques were used to analyze weight loss following infection. However, because each mouse was measured at every time point, mouse was included as a random effect in the regression model. Model simplification was performed in the publicly available software R 2.8.0. All remaining analyses were performed in JMP 4.0.4 (SAS Institute, Cary, N.C.).

### Example 2

### Innate Cell Recruitment During Highly Pathogenic and Sublethal Influenza Infections

While the expeditious accumulation of proinflammatory cytokines/chemokines during HP influenza infections has been extensively characterized, the identity of the cell types responsible for this immune-induced pathology has not been previously determined. Accordingly, lung recruitment kinetics were measured for natural killer cells (NK), conventional DCs (cDCs), neutrophils, macrophages, and tipDCs in C57BL/6J (B6) mice infected with either A/Puerto Rico/8/34 (PR8), a mouse adapted H1N1 virus, or x-31, a reassortant that contains the 6 internal genes of PR8 and the surface HA and NA from a prototypic H₃N₂ virus. Intranasal (i.n.) infection with 10³ plaque-forming units (pfu) of PR8 is uniformly lethal by day 10 after infection, whereas all those given 10⁵ pfu of x-31 recover by day 12 after losing between 15-20% of body weight. Tissues were collected daily (n=5) for 7 days after i.n. challenge and the inflammatory cell phenotypes were determined by flow cytometry. The trafficking profiles for macrophages, cDC, neutrophils, and NK cells were comparable for the two viruses. In contrast, the tipDCs accumulated to significantly higher numbers beginning day 3 and increased through to day 7 after infection with the highly pathogemic (HP) PR8 virus. Furthermore, proinflammatory cytokine/chemokine levels were relatively elevated in the bronchoalveolar lavage (BAL) supernatant from mice given the HP virus.

This analysis with x-31 and PR8 was then extended for two H5N1 influenza viruses: A/Vietnam/1203/2004 (VN1203, from a fatal human infection in 2004) having a LD₅₀ of 1 pfu in B6 mice, while A/Hong Kong/213/2003 (HK213) was much less virulent (LD₅₀=10^{3.7} pfu). Mice given 10² pfu of VN1203 were fatally compromised by day 9, whereas HK213 caused a 20% weight loss with full recovery by day 12. As before, the tipDCs were found at significantly higher cell counts in the lungs of the HP VN1203-infected animals, although there were no consistent differences in the numbers of macrophages, neutrophils, cDCs, or NK cells. Due to restrictions on the removal of samples from the high security BL3⁺ space where mice infected with these HP H5N1 viruses are held, cytokine analysis was not conducted.

### Example 3

### CCR2 Regulates Tipdc Trafficking and CD8⁺ T cell Response Magnitude

Emigration of tipDC precursors from the bone marrow is known to depend on signaling via the CCR2 receptor (Serbina & Pamer (2006) Nature Immunology 7(3):311). Thus, the trafficking kinetics of tipDCs between CCR2^{-/-} and CCR2^{+/+} (B6) mice were compared following infection with a sub-lethal dose (10² pfu) of PR8 virus. This analysis indicated that few, if any, tipDCs could be recovered from the lungs of the CCR2^{-/-} group. However, while greater tipDC numbers in the airways correlated with a more severe disease profile, unexpectedly CCR2^{-/-} mice showed no decrease in morbidity or mortality following HP virus challenge. Further analysis suggested that the continued vulnerability in the absence of tipDCs might reflect a failure to control the infection in the lung. While no virus could be recovered from the BAL fluid of the CCR2^{+/+} controls on day 9, a titer of 10^{3.6} pfu (±10^{3.3} s.e.m.) was recorded in comparable samples from the CCR2^{-/-} group. The primary mechanism for eliminating influenza virus-producing respiratory epithelial cells has previously been thought to be CD8⁺ cytotoxic T lymphocyte (CTL) mediated lysis. In addition to the effect on tipDC recruitment, there was (by staining with the D^{b}PA₂₂₄₋₂₃₃, K^{b}PB1₇₀₃₋₇₁₁ and D^{b}PB1-F2₆₂₋₇₀ tetramers) a substantial reduction in influenza-specific CD8⁺ CTL counts for BAL samples taken on day 7 after infection. This suggested an indirect effect, or that CTL numbers in some way were dependent on the concurrent presence of tipDCs. It seemed that the effect may be local, as it was only evident in the lungs and was not apparent in the regional mediastinal lymph nodes (MLN) where the virus-specific CD8⁺ T cell populations were of essentially equivalent magnitude in CCR2^{+/+} and CCR2^{-/-} mice.

### Example 4

### tipDCs Present Antigen to CD8 T-Cells in the Lungs

Because the influenza-specific CD8⁺ T cell counts were significantly reduced in the lungs (but not the lymph nodes) of CCR2^{-/-} animals, it was subsequently determined how tipDCs might regulate virus-specific CD8⁺ T cell numbers. One possibility was that the tipDCs could produce soluble factors that function to support CD8⁺ T cell survival and/or division. Therefore, the concentrations of cytokines/chemokines were determined in the cell-free BAL fluid from PR8-challenged CCR2^{-/-} and CCR2^{+/+} mice. The results of this analysis indicated that there were no significant differences for the key cytokines that are known to be involved in regulating CD8 T-cells, including IL-2 and IL-4. The next step was to assess the capacity of the tipDCs to function as antigen-presenting cells. For this experiment tipDCs (together with neutrophils as a control) were purified from the day 6 BAL wash of PR8-infected B6 mice, then incubated with a CD8⁺ T cell hybridoma specific for the D^{b}PB1-F2₆₂₋₇₀ epitope. While the neutrophils failed to stimulate the hybridoma, the freshly-isolated tipDCs presented the equivalent of 40 pmoles/cell of the D^{b}PB1-_{F262-70} epitope. As no exogenous peptide was added in this experiment, it was clear that the tipDCs had both processed influenza proteins in situ and expressed the PB1-F2₆₂₋₇₀ peptide/H2D^{b} MHC class I glycoprotein complex on the cell surface while in the virus-infected lung.

Demonstrating a capacity to process and present antigen that can be recognized by a CD8 T cell hybridoma does not establish, however, that tipDCs indeed serve as antigen presenting cells (APCs) during influenza virus infection. In fact, it was clear that tipDCs were not the sole APCs driving clonal expansion of influenza-specific CD8⁺ T cell precursors. Isolation of tipDCs from the draining MLN at any point after infection was not possible, and the lymph node CTL responses to the D^{b}PA₂₂₄₋₂₃₃, K^{b}PB1₇₀₃₋₇₁₁, and D^{b}PB1-F2₆₂₋₇₀ epitopes were equivalent for CCR2^{-/-} and CCR2^{+/+} mice. If tipDCs do serve as APCs in vivo, then this function is manifest outside the draining lymph node.

To further delineate if tipDCs could function as APCs within the respiratory tract, tipDCs were purified from the BAL of B6 mice that had been infected with either the x-31 or a double knock-out (DKO) x-31 virus. The mutated NP₃₆₆₋₃₇₄ and PA₂₂₄₋₂₃₃ peptides in the DKO virus do not complex with H2D^{b} and therefore the responses to these epitopes are lost (Webby, et al. (2003) Proc. Natl. Acad. Sci. USA 100(12):7235). Subsequently, 10⁴ tipDCs recovered from x-31 or DKO-infected mice were transferred directly into the airways of CCR2^{-/-} animals that had been infected with x-31 virus i.n. 24 hours previously. It was found that giving tipDCs from CCR2^{+/+} animals infected with the intact x-31 virus to CCR2^{-/-} animals was sufficient to recover the D^{b}NP₃₆₆₋₃₇₄ and D^{b}PA₂₂₄₋₂₃₃-specific CD8⁺ CTL responses, whereas those from comparable mice infected with the DKO virus were more than 10-fold less effective. The recovery of the CD8⁺ T cell response after tipDC transfer was clearly antigen-dependent, establishing that tipDCs indeed serve as APCs for CD8⁺ T cells in the lungs of mice infected with influenza A viruses. These results are in accord with a recent finding that CD8⁺ T cells and DCs interact in the lungs of influenza-infected mice (McGill, et al. (2008) J. Exp. Med. 205(7):1635-46).

### Example 5

### Reducing tipDC Accumulation Protects from Lethal Influenza Challenge

The data herein show that the number of tipDCs is significantly elevated in mice infected with HP influenza A viruses, that these tipDCs function locally in the respiratory tract as APCs, and that they are required for the full realization of protective CD8⁺ T cell-mediated immunity. In addition, their complete absence from the lungs of CCR2^{-/-} mice is associated with severe disease, indicating that tipDC ablation is not a viable therapeutic option.

Thus, it was contemplated that reducing, without eliminating, tipDC accumulation in the lungs following challenge with HP influenza A viruses may prove beneficial to the host. While searching for suppressors of CCL2 secretion, it was found that activation of the peroxisome proliferator-activated receptor-γ (PPARγ) with the synthetic agonist pioglitazone reduces the production of a wide range of proinflammatory molecules, including CCL2, TNF-α, and iNOS (Haraguchi, et al. (2008) Inten. Care Med. 34(7):1304). Accordingly, it was posited that a prophylactic regimen of pioglitazone may provide protection from HP influenza challenge by reducing the number of tipDCs recruited to the airways. Using weight loss as a measure for morbidity, B6 mice were treated with either pioglitazone (60 mg/kg in 100 μl PBS) or PBS (100 μl) via oral gavage daily beginning 3 days prior to infection with 5 MLD₅₀ of PR8 virus. The pioglitazone-treated animals were less severely affected and recovered more quickly than the PBS controls, while mortality was reduced from 92% to 50%.

To determine whether this protective effect was a consequence of decreased inflammation, immune cells within the lungs of mice treated with pioglitazone or PBS and challenged with 5 MLD₅₀ of PR8 virus were enumerated by flow cytometry. Although there was a general decrease in the magnitude of the cellular inflammatory exudate, the sole statistically significant reduction was in tipDC numbers. Furthermore, when the effect of pioglitazone on proinflammatory cytokines/chemokines in the BAL supernatant was measured, only CCL2 (MCP-1) and MCP-3 were reduced to a statistically significant level in the drug treated animals.

To determine if the protective effects of pioglitazone correlated with inhibition of virus replication, the virus titers in the BAL fluid from pioglitazone- and PBS-treated animals was measured on days 3, 6, and 9 after infection. No significant differences in virus titers were found at any time point. Further, treatment with up to 1 μg/ml pioglitazone (as compared to PBS) did not inhibit virus replication in madin-darby canine kidney (MDCK) cells. Together, these results indicate that the protection observed in pioglitazone-treated B6 mice reflects the reduced recruitment of tipDCs (via suppression of CCL2 (MCP-1) and MCP-3) rather than any inhibition of virus replication.

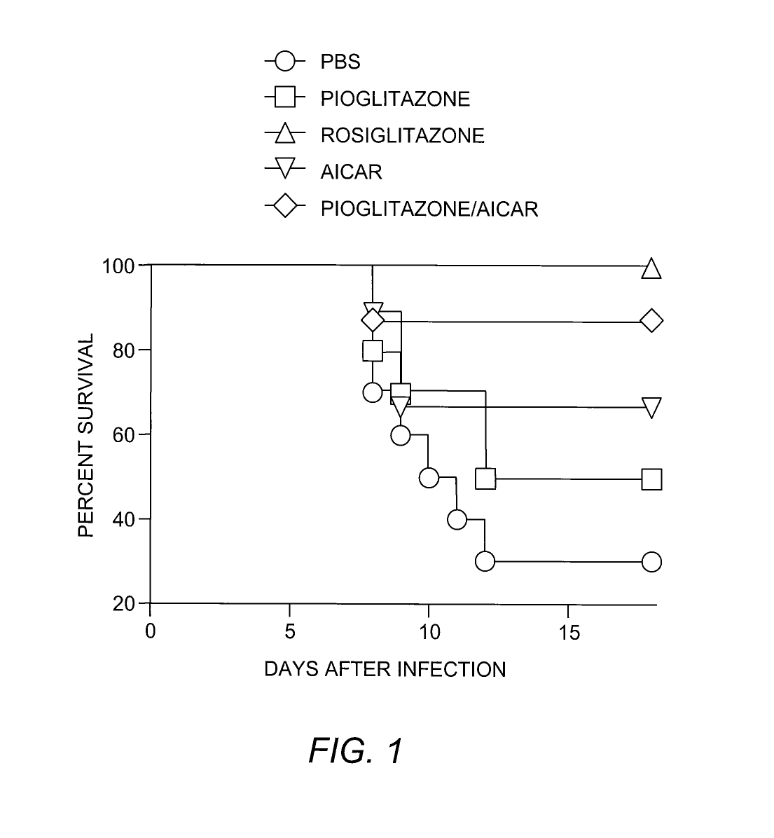

To further demonstrate protection afforded by PPAR agonism, mice were subjected to HP influenza challenge and treated with different PPAR agonists (Rosiglitazone or Pioglitazone), an agonist of the AMP Kinase upstream of PPAR signaling (AICAR), or a combination thereof (FIG. 1). The results of this analysis indicated that mice treated with PPAR agonists and/or AICAR exhibited reduced mortality to lethal influenza challenge compared to PBS controls. Indeed, the combination of Pioglitazone and AICAR was more effective than either agent alone. Of note, this combination is representative of the combination of Pioglitazone and metformin (an AMP kinase agonist), which is currently approved for use in the treatment of type 2 diabetes.

What is claimed is:

1. A method for preventing or ameliorating hypercytokinemia comprising administering to a subject in need of treatment an agent that suppresses TNF-α/Inducible nitric oxide synthase-Producing dendritic cell trafficking or accumulation thereby preventing or ameliorating hypercytokinemia.

2. The method of claim 1, wherein the agent reduces the expression or activity of MyD88, CCR2, MCP-1, or MCP-3.

3. The method of claim 2, wherein the agent is a peroxisome proliferator-activated receptor-γ agonist.

4. The method of claim 3, wherein the peroxisome proliferator-activated receptor-γ agonist is a thiazolidinedione.

5. The method of claim 4, wherein the thiazolidinedione is rosiglitazone, pioglitazone, troglitazone, rivoglitazone, ciglitazone, RS5444 or MCC-555.

6. The method of claim 1, wherein the subject has or is at risk of infection by a lethal influenza virus.

7. A method for reducing severity or protecting against lethality of an influenza A virus infection comprising administering to a subject in need of treatment an agent that suppresses TNF-α/Inducible nitric oxide synthase-Producing dendritic cell trafficking or accumulation thereby reducing the severity or protecting against lethality of influenza A virus infection.

8. The method of claim 7, wherein the agent reduces the expression or activity of MyD88, CCR2, MCP-1, or MCP-3.

9. The method of claim 8, wherein the agent is a peroxisome proliferator-activated receptor-γ agonist.

10. The method of claim 9, wherein the peroxisome proliferator-activated receptor-γ agonist is a thiazolidinedione.

11. The method of claim 10, wherein the thiazolidinedione is rosiglitazone, pioglitazone, troglitazone, rivoglitazone, ciglitazone, RS5444 or MCC-555.

12. A method for suppressing TNF-α/Inducible nitric oxide synthase-Producing dendritic cell trafficking or accumulation comprising administering to a subject in need of treatment a peroxisome proliferator-activated receptor-γ agonist thereby suppressing TNF-α/Inducible nitric oxide synthase-Producing dendritic cell trafficking or accumulation.

13. The method of claim 12, wherein the peroxisome proliferator-activated receptor-γ agonist is a thiazolidinedione.

14. The method of claim 13, wherein the thiazolidinedione is rosiglitazone, pioglitazone, troglitazone, rivoglitazone, ciglitazone, RS5444 or MCC-555.
